(19) United States
(12) Patent Application Publication
(10) Pub. No.: US 2026/0364132 A1
(43) Pub. Date: Nov. 19, 2026

(54) TOPICAL COMPOSITION

(71) Applicant: Sirenas LLC, San Diego, CA (US)

(72) Inventors: Eduardo Esquenazi, San Diego, CA (US); Elizabeth Paige Stout, San Diego, CA (US); Michael Howell, San Diego, CA (US); Tina Cheng, San Diego, CA (US); Tamara Schwent, Sarasota, FL (US)

(21) Appl. No.: 19/477,753

(22) PCT Filed: Oct. 3, 2024
(86) PCT No.: PCT/US2024/049852

§ 371 Date: Oct. 22, 2025

Related U.S. Application Data
(60) Provisional application No. 63/587,682, filed on Oct. 3, 2023.

(51) Int. Cl. A61K 36/03; A61K 31/01; A61K 31/195; A61K 31/455; A61K 36/72; A61K 36/73; A61K 36/754; A61K 36/886; A61P 29/00; A61P 37/02; A61P 39/06

(52) U.S. Cl. CPC A61K 36/03; A61K 31/01; A61K 31/195; A61K 31/455; A61K 36/72; A61K 36/73; A61K 36/754; A61K 36/886; A61P 29/00; A61P 37/02; A61P 39/06; A61K 2236/13; A61K 2236/15; A61K 2236/17; A61K 2236/333; A61K 2236/51; A61K 2236/53

(57) ABSTRACT

Topical compositions containing marine-sourced compounds are provided. The compositions may include a fucoidan-containing seaweed (e.g., Macrocystis pyrifera) extract, Evodia rutaecarpa extract, and optionally additional fucoidan. Methods of making and methods of using the topical compositions are also provided.

### FIELD

The present disclosure relates generally to the field of topical compositions, including compositions for the treatment and improvement of skin conditions.

### BACKGROUND

Sun, wind, and other environmental stressors can damage skin. Thus, there is a need for topical compositions that can repair damaged skin.

### SUMMARY

Disclosed herein is a composition comprising extracts from fucoidan-containing seaweeds and Evodia rutaecarpa extract.

In some embodiments, the composition further comprises an aloe extract. In some embodiments, the fucoidan-containing seaweed is Macrocystis pyrifera. In some embodiments, the composition further comprises additional fucoidan.

In some embodiments, the Macrocystis pyrifera extract is present in an amount between about 1% w/w and about 10% w/w. In some embodiments, the Macrocystis pyrifera extract is present in an amount between about 2% w/w and about 5% w/w. In some embodiments, the Macrocystis pyrifera extract is present in an amount between about 3% w/w and about 4% w/w. In some embodiments, the Macrocystis pyrifera extract is present in an amount between about 0.001% w/w and about 0.9% w/w. In some embodiments, the Macrocystis pyrifera extract is present in an amount between about 0.02% w/w and about 0.15% w/w.

In some embodiments, the Evodia rutaecarpa extract is present in an amount between about 2% w/w and about 20% w/w. In some embodiments, the Evodia rutaecarpa extract is present in an amount between about 5% w/w and about 10% w/w. In some embodiments, the Evodia rutaecarpa extract is present in an amount between about 7% w/w and 8% w/w. In some embodiments, the Evodia rutaecarpa extract is present in an amount between about 0.001% w/w and about 1% w/w. In some embodiments, the Evodia rutaecarpa extract is present in an amount between about 0.05% w/w and about 0.3% w/w. In some embodiments, the Evodia rutaecarpa extract comprises a solution in propane-1,3-diol. In some embodiments, the Evodia rutaecarpa extract comprises a solution in propanediol.

In some embodiments, the additional fucoidan is in an extract from a seaweed present in an amount between about 2% w/w and about 20% w/w. In some embodiments, the additional fucoidan is in an extract from a seaweed present in an amount between about 5% w/w and about 10% w/w. In some embodiments, the additional fucoidan is in an extract from a seaweed present in an amount between about 7% w/w and about 8% w/w. In some embodiments, the additional fucoidan is in an extract from a seaweed present in an amount between about 0.001% w/w and about 1% w/w. In some embodiments, the additional fucoidan is in an extract from a seaweed present in an amount between about 0.1% w/w and about 0.5% w/w.

In some embodiments, rutaecarpine is present in an amount between about 15 mg/kg to about 200 mg/kg. In some embodiments, rutaecarpine is present in an amount between about 30 mg/kg to about 80 mg/kg. In some embodiments, rutaecarpine is present in an amount between about 50 mg/kg to about 60 mg/kg. In some embodiments, rutaecarpine is present in an amount between about 0.01 mg/kg to about 10 mg/kg. In some embodiments, rutaecarpine is present in an amount between about 0.1 mg/kg to about 5 mg/kg.

In some embodiments, evodiamine is present in an amount between about 20 mg/kg to about 300 mg/kg. In some embodiments, evodiamine is present in an amount between about 40 mg/kg to about 150 mg/kg. In some embodiments, evodiamine is present in an amount between about 80 mg/kg to about 110 mg/kg. In some embodiments, evodiamine is present in an amount between about 0.01 mg/kg to about 10 mg/kg. In some embodiments, evodiamine is present in an amount between about 0.1 mg/kg to about 5 mg/kg.

In some embodiments, fucoxanthin is present in an amount between about 5 mg/kg to about 400 mg/kg. In some embodiments, fucoxanthin is present in an amount between about 10 mg/kg to about 200 mg/kg. In some embodiments, fucoxanthin is present in an amount between about 50 mg/kg to about 100 mg/kg. In some embodiments, fucoxanthin is present in an amount between about 0.01 mg/kg to about 20 mg/kg. In some embodiments, fucoxanthin is present in an amount between about 0.1 mg/kg to about 6 mg/kg.

In some embodiments, the composition is in the form of: lotion, spray, cream, serum, aqueous solution, paste, jelly, wax, oil, semi-solid gel, ointment, gel-cream, lip balm, or multi-purpose balm.

In some embodiments, the composition further comprises one or more of: propane-1,3-diol, shea butter, cocoa butter, olive oil, coconut oil, avocado oil, dimethicone, petroleum jelly, glycerin, ceramide, honey, buttermilk, castor oil, aloe extract, aloe vera gel, aloe butter, argan oil, caprylic/capric triglycerides, flaxseed oil, jojoba oil, rose him seed oil, safflower oil, sunflower oil, sea buckthorn oil, red raspberry seed oil, squalane, niacinamide, mango butter, kokum butter, almond butter, apricot kernel butter, hemp butter, macadamia nut butter, sal butter, beeswax, black cumin seed oil, vitamin E oil, murumuru butter, cupuacu butter, tamanu oil, meadowfoam seed oil, candelilla wax, carnauba wax, laurel wax, sunflower seed wax, and Cera bellina.

In some embodiments, the composition further comprises propane-1,3-diol. In some embodiments, the composition further comprises propanediol. In some embodiments, the composition further comprises about 15% w/w aloe extract. In some embodiments, the composition further comprises about 20% w/w aloe vera gel. In some embodiments, the composition further comprises about 3% w/w to about 5% w/w sea buckthorn oil. In some embodiments, the composition further comprises about 0.5% w/w to about 5% w/w sea buckthorn oil. In some embodiments, the composition further comprises about 5% w/w to about 10% w/w squalane. In some embodiments, the composition further comprises about 0.5% w/w to about 10% w/w squalane. In some embodiments, the composition further comprises about 3% w/w to about 5% w/w red raspberry seed oil. In some embodiments, the composition further comprises about 3% w/w to about 5% w/w niacinamide. In some embodiments, the composition further comprises about 1% w/w to about 10% w/w niacinamide. In some embodiments, the composition further comprises about 0.5% w/w to about 5% w/w panthenol (pro-vitamin B5).

Also disclosed herein is a composition comprising rutaecarpine, evodiamine, and fucoxanthin.

In some embodiments, rutaecarpine is present in an amount between about 30 mg/kg to about 80 mg/kg. In some embodiments, rutaecarpine is present in an amount between about 0.01 mg/kg to about 10 mg/kg.

In some embodiments, evodiamine is present in an amount between about 40 mg/kg to about 150 mg/kg. In some embodiments, evodiamine is present in an amount between about 0.01 mg/kg to about 10 mg/kg.

In some embodiments, fucoxanthin is present in an amount between about 10 mg/kg to about 200 mg/kg. In some embodiments, fucoxanthin is present in an amount between about 0.05 mg/kg to about 10 mg/kg.

Also disclosed herein is a method of manufacturing a composition comprising the steps of:
a) combining Macrocystis pyrifera powder with ethanol and water to create a first mixture;
b) filtering the first mixture to remove undissolved solids; and
c) evaporating the ethanol and water from the first mixture to generate a solid Macrocystis pyrifera extract.

In some embodiments, the ethanol and water are present in a ratio of about 4:1. In some embodiments, the method further comprises bringing the solid extract into contact with Evodia rutaecarpa extract to form a second mixture. In some embodiments, the Evodia rutaecarpa extract comprises a solvent. In some embodiments, the solvent is propane-1,3-diol. In some embodiments, the solvent is propanediol.

In some embodiments, the method further comprises filtering the second mixture to remove undissolved solids. In some embodiments, the method further comprises combining fucoidan with the second mixture.

Also disclosed herein is a composition prepared by a method as described herein.

In another embodiment provided herein is a method of making a skin care product, comprising combining a composition as described herein with a carrier. In some embodiments, the carrier is shea butter. In some embodiments, the carrier is caprylic/capric triglycerides. In some embodiments, the carrier is sunflower seed oil, jojoba oil, or squalane.

In yet another embodiment provided herein is method of treating a condition of the skin, comprising administering a composition as described herein to the skin of the subject.

In some embodiments, the condition is selected from the group consisting of: sunburn, windburn, inflammation, irritation, eczema, psoriasis, wrinkles, sagging skin, acne, rosacea, damage related to environmental exposure, and damage related to pollution and toxins.

Some embodiments include a method of reducing oxidative stress, comprising administering the composition to the skin of a subject. Some embodiments include a method of reducing UV-induced inflammation, comprising administering the composition to the skin of a subject.

In some embodiments, about 0.1 ml to about 0.2 ml of the composition is administered onto the skin per dose. In some embodiments, about 0.4 ml to about 0.6 ml of the composition is administered onto the skin per dose. In some embodiments, about 1.0 ml to about 1.5 ml of the composition is administered onto the skin per dose.

In some embodiments, the composition is administered once. In some embodiments, the composition is administered once a day. In some embodiments, the composition is administered twice a day. In some embodiments, the composition is administered every 4 hours. In some embodiments, the duration of administration is 1 day. In some embodiments, the duration of administration is 2 weeks. In some embodiments, the duration of administration is 60 days. In some embodiments, the duration of administration is 4 months.

In some embodiments, the composition is administered to a surface area on the skin of about 0.8 cm². In some embodiments, the composition is administered to a surface area on the skin of about 14 in². In some embodiments, the composition is administered to a surface area on the skin of about 25 in².

In some embodiments, the composition comprises a deodorant. In some embodiments, the composition comprises a moisturizer. In some embodiments, the composition comprises a balm. In some embodiments, the composition comprises a deodorant balm.

### BRIEF DESCRIPTION OF THE DRAWINGS

FIG. 1 shows the mass spectral data for the base composition.

FIGS. 2A and 2B show the mass spectral data for Peak 1 of the spectrum in FIG. 1 and the chemical structure of evodiamine, respectively.

FIGS. 3A and 3B show the mass spectral data for Peak 2 of the spectrum in FIG. 1 and the chemical structure of rutaecarpine, respectively.

FIG. 4 is a graph showing the antioxidant activity of selected compositions.

### DETAILED DESCRIPTION

Embodiments provided herein relate to topical formulations for application to skin. In some embodiments, the topical formulations comprise naturally sourced compounds.

It will be readily understood that the aspects of the present disclosure, as generally described herein, can be arranged, substituted, combined, separated, and designed in a wide variety of different configurations, all of which are explicitly contemplated herein.

### DEFINITIONS

Unless defined otherwise, technical, and scientific terms used herein have the same meaning as commonly understood by one of ordinary skill in the art to which the present disclosure belongs.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “about” is meant a quantity, level, value, number, frequency, percentage, dimension, size, amount, weight, or length that varies by as much as 30, 25, 20, 15, 10, 9, 8, 7, 6, 5, 4, 3, 2, 1, 0.25, or 0.1% to a reference quantity, level, value, number, frequency, percentage, dimension, size, amount, weight, or length.

As used herein, a “subject” or a “patient” refers to an animal that is the object of treatment. “Animal” includes, but is not limited to, mammals. “Mammal” comprises, without limitation, mice, rats, rabbits, guinea pigs, dogs, cats, sheep, goats, cows, horses, primates, such as monkeys (e.g., rhesus and macaque), chimpanzees, and apes, and, in particular, humans. In some alternatives, the subject is human.

Some embodiments disclosed herein relate to selecting a subject or patient in need. In some embodiments, a patient is selected who is in need of treatment, amelioration, inhibition, progression, prophylaxis, or improvement in disease symptoms or who is in need of curative therapy.

As used herein, the term “treatment” refers to an intervention made in response to a disease, disorder, or physiological condition manifested by a subject. The terms treating, treatment, therapeutic, or therapy do not necessarily mean total cure or abolition of the disease or condition. For example, in some embodiments, treatments reduce, alleviate, ameliorate, or eradicate the symptom(s) of the disease and/or provide curative therapy of the disease.

The term “therapeutically effective amount” is used to indicate an amount of a composition that elicits the biological or medicinal response indicated. Determination of a therapeutically effective amount is within the capability of those skilled in the art, in view of the disclosure provided herein. The therapeutically effective amount of the composition disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication, and other factors which those skilled in the medical arts will recognize.

As used herein, “Macrocystis pyrifera extract” is a collection of chemical compounds removed from Macrocystis pyrifera. In some embodiments, the extract comprises fucoidan.

As used herein, “Fucus vesiculosus extract” is a collection of chemical compounds removed from Fucus vesiculosus. In some embodiments, the extract comprises fucoidan.

As used herein, “Undaria pinnatifida extract” is a collection of chemical compounds removed from Undaria pinnatifida. In some embodiments, the extract comprises fucoidan.

As used herein, “Evodia rutaecarpa extract” is a collection of chemical compounds removed from Evodia rutaecarpa. In some embodiments, the extract comprises rutaecarpine and evodiamine.

As used herein, “Laminaria extract” is a collection of chemical compounds removed from Laminaria spp, including but not limited to Laminaria digitata, Laminaria saccharine, and Laminaria japonica.

As used herein, an “excipient” is a substance formulated alongside the other ingredients, included for the purpose of improving stabilization of the composition, bulking up the composition, or to confer an enhancement on the effects of one or more other ingredients in the composition. An excipient may be an adherent, a binder, a coating, a color, a disintegrant, a flavor, a glidant, a lubricant, a preservative, a sorbent, a sweetener, or a vehicle.

As used herein, the term “coadministration” of pharmacologically active compounds refers to the delivery of two or more separate chemical entities or separate therapies, whether in vitro or in vivo. Coadministration refers to the simultaneous delivery of separate agents or therapies; to the simultaneous delivery of a mixture of agents; to the delivery of one agent followed by delivery of a second agent or additional agents; or to the administration of one therapy followed by or concomitant with another therapy. In all cases, agents or therapies that are coadministered are intended to work in conjunction with each other. Similarly, in the context of administration of more than one compound, the term “in combination” refers to a concomitant delivery of one compound with one or more compounds. The compounds may be administered in combination by simultaneous administration or administration of one compound before or after administration of another compound.

As used herein, “topical” refers to the administration or application of a formulation to the skin or various body orifices.

The disclosure is generally described herein using affirmative language to describe the numerous embodiments. The disclosure also includes embodiments in which subject matter is excluded, in full or in part, such as substances or materials, method steps and conditions, protocols, or procedures.

### Base Composition

Some embodiments provide a base composition comprising a combination of Evodia rutaecarpa extract and an extract from a fucoidan-containing seaweed. The base composition may be used as a starting composition to prepare various formulations for topical administration. Alternatively, the base composition may be used directly for topical administration. In some embodiments, the base composition is provided as solid composition, such as a dry powder composition. In other embodiments, the base composition is provided as a solution in a suitable solvent. The solvent may include water, ethanol, propanediol, propane-1,3-diol, DMSO, methylcellulose, hydrogel, caprylic/capric triglycerides, coco-caprylate/caprate, sunflower seed oil, jojoba oil, squalane, a combination thereof, or other suitable solvent.

The amount of Evodia rutaecarpa extract included in the base composition may be from about 1% w/w to about 20% w/w, about 1% w/w to about 10% w/w, about 5% w/w to about 10% w/w or about 2% w/w to about 5% w/w. The amount of fucoidan-containing seaweed extract included in the base composition may be from about 1% w/w to about 30% w/w, about 2% w/w to about 20% w/w, about 2% w/w to about 5% w/w, or about 5% w/w to about 10% w/w. In some embodiments the fucoidan-containing seaweed is selected from Macrocystis pyrifera, Fucus vesiculosus, Undaria pinnatifida, Laminaria spp. (e.g., Laminaria digitata, Laminaria saccharine, and Laminaria japonica), and a combination thereof.

In some embodiments, the base composition may be supplemented with additional fucoidan, for example extracted and purified from Macrocystis pyrifera, Fucus vesiculosus, Undaria pinnatifida, Laminaria spp. (e.g., Laminaria digitata, Laminaria saccharine, and Laminaria japonica), or another seaweed species. In some embodiments, the additional fucoidan is in an extract from Undaria pinnatifida. The additional fucoidan-containing extract may either be included in the base composition, or added directly to the final formulation. When included in the base composition, the additional fucoidan-containing extract may be from about 0.1% w/w/to about 20% w/w, from about 0.1% w/w/to about 15% w/w, from about 0.1% w/w/to about 10% w/w. One preferred specification for the base composition is provided in Table 1.

TABLE 1
Components of the Base Composition

| | |
|---|---|
| Component | Amount |

| | | |
|---|---|---|
| propane-1,3-diol | 70-100% | (w/w %) |
| Evodia rutaecarpa extract | 0.1-10% | (w/w %) |
| Macrocystis pyrifera extract | 0.1-5% | (w/w %) |
| Optional additional fucoidan extract | 0.1-10% | (w/w %) |
| (e.g., Undaria pinnatifida extract) | | |

The weight ratio of Evodia rutaecarpa extract to fucoidan-containing seaweed extract (e.g., Macrocystis pyrifera extract) in the base composition may range from about 20:1 to about 1:20, from about 10:1 to about 1:10, from about 10:1 to about 1:5, from about 5:1 to about 1:2, or from about 5:1 to about 1:1. When an additional fucoidan-containing extract is included, the weight ratio of Evodia rutaecarpa extract to the additional fucoidan-containing extract (e.g., Undaria pinnatifida extract) may range from about 10:1 to about 1:20, from about 10:1 to about 1:15, from about 5:1 to about 1:10, or from about 3:1 to 1:10. Some embodiments include Evodia rutaecarpa extract, Macrocystis pyrifera extract, and Undaria pinnatifida extract at a weight ratio of 1-100:0.5-50:1-100, 2-50:1-30:5-75, or 5-30:2-15:10-50 (Evodia rutaecarpa:Macrocystis pyrifera:Undaria pinnatifida).

In some embodiments, the base composition may be characterized by the presence of rutaecarpine, evodiamine, fucoidan, and fucoxanthin, which may be provided by Evodia rutaecarpa and Macrocystis pyrifera extracts, or from other sources. In some embodiments, the amount of rutaecarpine in the base composition is from about 10 mg/kg to about 150 mg/kg, from about 20 mg/kg to about 100 mg/kg, or about 30 mg/kg to about 80 mg/kg. In some embodiments, the amount of evodiamine in the base composition is from about 10 mg/kg to about 200 mg/kg, from about 20 mg/kg to about 120 mg/kg, or about 40 mg/kg to about 150 mg/kg. In some embodiments, the amount of fucoxanthin in the base composition is from about 2 mg/kg to about 400 mg/kg, from about 5 mg/kg to about 300 mg/kg, or about 10 mg/kg to about 200 mg/kg.

In some embodiments, the weight ratio of rutaecarpine to evodiamine in the base composition is from about 1:2 to about 1:20, from about 1:1 to about 1:10, or from about 2:1 to about 1:5.

The base composition may be prepared by the following procedure. Fucoidan-containing seaweed (e.g., Macrocystis pyrifera) powder is combined with a mixture of ethanol and water. The ratio of ethanol and water may be 10:1, 9:1, 8:1, 7:1, 6:1, 5:1, 4:1, 3:1, 2:1, or 1:1, or a range between any two of these values. The resulting mixture is stirred at rt. The mixture may be stirred for 1 hour, 2 hours, 4 hours, 8 hours, 12 hours, 16 hours, 24 hours, or 36 hours, or a range between any two of these values. The mixture is then filtered, and the filtrate is collected. The solvent is removed from the filtrate, resulting in a dried extract. The extract is subsequently brought up in another mixture of water and ethanol, and the steps of stirring, filtering, and drying the solvent are repeated. The extract is subjected to these steps 1, 2, 3, 4, 5, 6, 7, 8, 9, or 10 times. The extract may be stored in a vial at rt.

The resulting extract is then combined with Evodia rutaecarpa extract in propane-1,3-diol. When combined, the resulting concentration of fucoidan-containing seaweed (e.g., Macrocystis Pyrifera) extract may be between 20-40 mg/mL (w/v). The mixture is sonicated at rt for 1, 5, 10, 15, 30, 60, or 90 minutes, or a range between any two of these values. Next, the mixture is stirred at rt for 5, 15, 30, 45, 60, or 90 minutes, or a range between any two of these values. The mixture is filtered, and the filtrate is collected. The filtrate may be combined with fucoidan dissolved in distilled water, resulting in a concentration from 5-20 mg/ml. The aqueous fucoidan solution may be added to the propane-1,3-diol solution up to 10% (v/v).

### Formulations

In some embodiments, the base composition may be combined with other ingredients to form a new topical composition. In some embodiments, the base composition may be combined with one or more of the following ingredients: propane-1,3-diol, shea butter, cocoa butter, olive oil, coconut oil, avocado oil, dimethicone, petroleum jelly, glycerin, ceramide, honey, buttermilk, castor oil, aloe extract, aloe vera gel, aloe butter, argan oil, caprylic/capric triglycerides, flaxseed oil, jojoba oil, rose hip seed oil, safflower oil, sunflower oil, sea buckthorn oil, red raspberry seed oil, squalane, niacinamide, mango butter, kokum butter, almond butter, apricot kernel butter, hemp butter, macadamia nut butter, sal butter, beeswax, black cumin seed oil, and vitamin E oil.

In various topical formulations prepared by combination of the base composition with other ingredients, the amount of base composition, including its solvent, in the formulation may be from about 0.1% w/w to about 20% w/w, from about 0.5% w/w to about 10% w/w, from about 1% w/w to about 5% w/w, or from about 1% w/w to about 3% w/w.

For example, a topical composition may comprise 1% w/w base composition, at least 15% w/w aloe extract, 3-5% w/w red raspberry seed oil, and 3-5% w/w niacinamide. In another embodiment, a topical composition may comprise 3% w/w base composition, at least 20% w/w aloe vera gel, 3-5% w/w sea buckthorn oil, and 5-10% w/w squalane.

In some embodiments, the amount of Evodia rutaecarpa extract in the final topical formulation may be from about 0.0005% w/w to about 1% w/w, about 0.001% w/w to about 0.5% w/w, about 0.05% w/w to about 0.3% w/w, about 0.05% w/w to about 0.1% w/w, or about 0.15% w/w to about 0.3% w/w. The amount of fucoidan-containing seaweed (e.g., Macrocystis pyrifera) extract included in the final topical formulation may be from about 0.001% w/w to about 0.9% w/w, about 0.05% w/w to about 0.5% w/w, about 0.01% w/w to about 0.2% w/w, about 0.02% w/w to about 0.15% w/w, about 0.02% w/w to about 0.05% w/w, or about 0.06% w/w to about 0.15% w/w. If present, the amount of additional fucoidan-containing extract (e.g., Undaria pinnatifida extract), provided either as part of the base composition or separately included in the final topical formulation, may be from about 0.01% w/w to about 2% w/w, from about 0.05% w/w to about 1% w/w, or about 0.1% w/w to about 0.5% w/w. It will be appreciated that the weight ratios of the extracts in the final topical formulation will be the same as the extracts that are included in the base composition. Thus, for example, the weight ratios of the extracts in the final topical formulation can be 1-100:0.5-50:1-100, 2-50:1-30:5-75, or 5-30:2-15:10-50 (Evodia rutaecarpa:Macrocystis pyrifera:Undaria pinnatifida).

Some embodiments include a topical formulation comprising rutaecarpine and fucoidan. In some embodiments, the amount of rutaecarpine in the final topical formulation may be from about 0.01 mg/kg to about 20 mg/kg, from about 0.05 mg/kg to about 10 mg/kg, or about 0.1 mg/kg to about 5 mg/kg. In some embodiments, the amount of evodiamine in the final topical formulation may be from about 0.01 mg/kg to about 20 mg/kg, from about 0.05 mg/kg to about 10 mg/kg, or about 0.1 mg/kg to about 5 mg/kg. In some embodiments, the amount of fucoxanthin in the final topical formulation may be from about 0.01 mg/kg to about 20 mg/kg, from about 0.05 mg/kg to about 10 mg/kg, or about 0.1 mg/kg to about 6 mg/kg.

The formulations provided herein may be prepared, packaged, or sold for topical administration. The formulations can be filled into suitable packaging (containers) such as, for example, tubes, cartons, capsule, jars, bottles, canisters, squeeze pack, pouches, packages, packets, pumps, sacks, tank, or other containers. In some embodiments, the formulation may be applied directly to skin. In some embodiments, the formulation may be applied by an applicator, a brush, or other device for application to the skin.

Packaging can include single use aliquots in single use packaging such as pouches. The formulation can be packaged in suitable packaging having volumes of about 0.1 oz., about 0.2 oz., about 0.5 oz., about 1 oz., about 2 oz., about 3.4 oz., about 4 oz., about 8 oz., about 16 oz., about 32 oz., about 48 oz., about 64 oz., about 80 oz., about 96 oz., about 112 oz., about 128 oz., about 144 oz., about 160 oz., or an amount within a range defined by any two of the aforementioned values. The packaging can also be squeezable pouches having similar volumes. In some embodiments, packaging may be free of dyes, metal specks, or chemicals that can be dissolved by acids or oxidizing agents. In other embodiments, any bottles, package caps, bottling filters, valves, lines, and heads used in packaging may be specifically rated for acids and oxidizing agents. In some cases, package caps with any organic glues, seals, or other components sensitive to oxidation may be avoided since they could neutralize and weaken the product over time.

In some embodiments, the active ingredients and mixtures of active ingredients can be used, for example, in topical formulations including a pharmaceutically acceptable carrier prepared for storage and subsequent administration.

Preservatives can be used to keep the compounds in the formulations described herein from breaking down. As used herein, the terms “carrier or diluent” may be a solid carrier or diluent for solid formulations, a liquid carrier or diluent for liquid formulations, or mixtures thereof. Solid carriers/diluents include, but are not limited to, shea butter, a gum, a starch (e.g., corn starch, pregeletanized starch), a sugar (e.g., lactose, mannitol, sucrose, dextrose), a cellulosic material (e.g., microcrystalline cellulose), an acrylate (e.g., polymethacrylate), calcium carbonate, magnesium oxide, talc, or mixtures thereof. For liquid formulations, such as for topical or parenteral formulations, acceptable carriers may be aqueous or non-aqueous solutions, suspensions, emulsions, or oils. Examples of non-aqueous solvents are caprylic/capric triglycerides, propylene glycol, polyethylene glycol, and injectable organic esters such as ethyl oleate. Aqueous carriers include water, alcoholic/aqueous solutions, emulsions, or suspensions, including saline and buffered media. Examples of oils are those of petroleum, animal, vegetable, or synthetic origin, for example, almond oil, peanut oil, soybean oil, mineral oil, olive oil, sunflower oil, and fish-liver oil.

### Disorders

As provided herein, a topical formulation which may be used in treating a subject in need is provided. The subject in need can have diseases, disorders, ailments, and or damage of skin, hair, and/or nails, including skin damage, hair and scalp disorders, and nail disease. In some embodiments, the subject suffers from inflammation. In some embodiments, the subject can have a wound. In some embodiments, the subject suffers from skin irritation. In some embodiments, the subject can suffer from skin damage due to environmental exposure or pollution and toxins. In some embodiments, the subject suffers from a disrupted or damaged skin barrier. In some embodiments, the subject suffers from hyperpigmentation or other skin pigmentation disorders. In some embodiments, the skin pigmentation disorder is one or more of albinism, pigment loss after skin damage, and vitiligo. In some embodiments, a topical formulation described herein is used as an anti-aging agent.

“Skin damage” as described herein, can refer to damage to the skin that can be caused by aging, sun damage, cancer, skin disorder or skin diseases that can cause irritation of the skin. Without being limiting, the “skin diseases” and/or “skin disorders” can include rhytide, non-enzymatic glycosylation of the skin, sun damage, smoking damage, fibrosis of the skin, acne aestivalis (Mallorca acne), acne conglobate, acne cosmetica (cosmetic acne), acne fulminans (acute febrile ulcerative acne), acne keloidalis nuchae (acne keloidalis, dermatitis papillaris capillitii, folliculitis keloidalis, folliculitis keloidis nuchae, nuchal keloid acne), adult forehead with scattered red pimples, acne vulgaris, acne mechanica, acne medicamentosa, acne miliaris necrotica (acne varioliformis), acne vulgaris, acne with facial edema (solid facial edema), blepharophyma, contact dermatitis, cutaneous graft-versus-host disease, eczema, erythrotelangiectatic rosacea (erythematotelangiectatic rosacea, vascular rosacea), excoriated acne (acne excoriee des jeunes filles, Picker's acne), glandular rosacea, gnathophyma, gram-negative rosacea, granulomatous facial dermatitis, adult male with a large, red, bulbous nose, rhinophyma, granulomatous perioral dermatitis, halogen acne, hidradenitis suppurativa (acne inversa, pyoderma fistulans significa, Verneuil's disease), ichthyosis, idiopathic facial aseptic granuloma, infantile acne, irritant dermatitis, lichen planus, lupoid rosacea (granulomatous rosacea, micropapular tuberculid, rosacea-like tuberculid of Lewandowsky), lupus miliaris disseminatus faciei, metophyma, neonatal acne (acne infantum, acne neonatorum, neonatal cephalic pustulosis), occupational acne, oil acne, ocular rosacea (ophthalmic rosacea, ophthalmorosacea), otophyma, periorificial dermatitis, persistent edema of rosacea (chronic upper facial erythematous edema, Morbihan's disease, rosaceous lymphedema), phymatous rosacea, pomade acne, papulopustular rosacea (inflammatory rosacea), perifolliculitis capitis abscedens et suffodiens (dissecting cellulitis of the scalp, dissecting folliculitis, perifolliculitis capitis abscedens et suffodiens of Hoffman), perioral dermatitis, periorbital dermatitis (periocular dermatitis), pyoderma faciale (rosacea fulminans), radiation dermatitis, rhinophyma, rosacea (acne rosacea), rosacea conglobate, synovitis-acne-pustulosis-hyperostosis-osteomyelitis syndrome (SAPHO syndrome), steroid rosacea, tar acne, skin cancer, tropical acne, psoriasis, including plaque psoriasis, guttate psoriasis, inverse psoriasis, pustular psoriasis, erythrodermic psoriasis, nail psoriasis, psoriatic arthritis, and combinations and/or variations thereof. In some embodiments described herein, a method of treating a subject in need is provided. The subject can have a disease affecting the skin as provided described herein.

“Hair and scalp disorders” are diseases that affect the hair and scalp and are also described herein. Diseases that affect hair and scalp can include but are not limited to alopecia, androgenic alopecia, hirsutism, hair shaft disorders, inflammation, acromegaly, eczema, psoriasis, impetigo, atopic dermatitis, darier disease, and folliculitis. Common causes for scalp disorders can include but are not limited to acromegaly, atopic dermatitis, darier disease, eczema, fragile X syndrome, impetigo, pachydermoperiostosis, psoriasis, and Rosenthal-Kloepfer syndrome. In some embodiments described herein, a method of treating a subject in need is provided. The subject can have a disease affecting the skin and scalp. In some embodiments the subject suffers from alopecia, androgenic alopecia, hirsutism, hair shaft disorders, inflammation, acromegaly, eczema, psoriasis, impetigo, atopic dermatitis, darier disease, and/orfolliculitis. In some embodiments, the subject suffers from acromegaly, atopic dermatitis, darier disease, eczema, fragile X syndrome, impetigo, pachydermoperiostosis, psoriasis, and/or Rosenthal-Kloepfer syndrome. In some embodiments, the treating includes administering a formulation to the subject in need. In some embodiments, the formulation is within a hair cream, a hair gel, a scalp lotion, a shampoo, conditioner, hair spray, or a hair mousse.

“Nail diseases” are disorders or diseases that affect the nail, nail bed, or cuticle region and are also described herein. Diseases that affect the nail and surrounding skin area such as the cuticle can lead to infection or inflammation that could require medical assistance. Diseases that infect the nail, nail bed, and/or cuticle can include but is not limited to onychia, onchyocryptosis, onychodystophy, onychogryposis, onycholysis, onychomadesis, onychomycosis, tinea unguium, onychophosis, onychoptosis, onchorrhexis, paronychia, Koilonychia, subungual hematoma, onychomatricoma, nail pemphigus, erythronychia, and melanonychia. In some embodiments described herein, a method of treating a subject in need is provided. The subject can have a disease affecting the nails, nail bed, and/or cuticles. In some embodiments, the subject suffers from onychia, onchyocryptosis, onychodystophy, onychogryposis, onycholysis, onychomadesis, onychomycosis, tinea unguium, onychophosis, onychoptosis, onchorrhexis, paronychia, Koilonychia, subungual hematoma, onychomatricoma, nail pemphigus, erythronychia, and/or melanonychia. In some embodiments, the treating includes administering a formulation to the subject in need. In some embodiments, the formulation is within a skin cream, a lotion, a cuticle cream, or a nail polish.

“Inflammation” as described herein, refers to a biological response of a body tissue to harmful stimuli. The harmful stimuli can include but is not limited to ultraviolet light, pathogens, bacteria, viruses, fungi, damaged cells, and other irritants that are known to those skilled in the art. Inflammation can be a protective immune response that can involve, for example, immune cells, white blood cells, blood vessels, molecular mediators, and other small molecules. Signs of inflammation can include but is not limited to pain, heat, swelling, and/or loss of function. Inflammation can be acute or chronic. In some embodiments described herein, a formulation is provided for the treatment of inflammation. In some embodiments, the subject suffers from inflammation. In some embodiments, the inflammation is on the skin, scalp, nasal passages, mouth, nail area such as the cuticles, eyes, vaginal area, or the perineal area.

“Anti-aging” as described herein, refers to preventing or lessening the appearance of getting older. Collagen and elastin break down in the skin over time, causing it to become looser and more prone to wrinkling. Exposure to ultraviolet light, smoking, and drinking alcohol break down collagen further. Repetitive facial expressions such as squinting or frowning can exacerbate the appearance of wrinkles. Anti-aging activity may slow down the signs of aging and improve skin's appearance.

### EXAMPLES

Some aspects of the embodiments discussed above are disclosed in further detail in the following examples, which are not in any way intended to limit the scope of the present disclosure. Those in the art will appreciate that many other embodiments also fall within the scope of the disclosure, as it is described herein above and in the claims.

The following abbreviations have the indicated meanings:
DMEM=Dulbecco's Modified Eagle Medium
DMSO=dimethylsulfoxide
DPPH=2,2-Diphenyl-1-(2,4,6-trinitrophenyl)hydrazin-1-yl
Et=ethyl
H2DCFDA=2′,7′-Dichlorodihydrofluorescein diacetate
HPLC=high-performance liquid chromatography
MEM=Minimum Essential Medium
RT=room temperature

### Example 1. Preparation of Base Composition

The following example describes an embodiment of a base composition and the method of making the composition. Information related to starting materials used in the preparation of the composition is as follows.

Undaria pinnatifida Extraction Method

Freshly collected Undaria pinnatifida is washed thoroughly with distilled water and dried in an oven at 40 °C. The dried U. pinnatifida is ground into a fine powder. The powder is then added to distilled water (1:20 w/v) and heated to 70 °C with stirring overnight. The heterogeneous mixture is then centrifuged to separate solids from the liquid extract. Alginate is removed by adding 2% CaCl₂) to the liquid extract and stored at 4 °C overnight. Alginate precipitate is removed through centrifugation. Anhydrous ethanol (EtOH) is added to the supernatant to reach 70% EtOH concentration. A precipitated fucoidan-containing composition (>85% fucoidan) is collected after centrifugation. The precipitate is placed in a freeze-dryer to obtain crude fucoidan-containing powder.

Evodia rutaecarpa Extraction Method

Evodia rutaecarpa powder is added to an Erlenmeyer flask equipped with a stir bar. 95% Ethanol:Water (EtOH:H₂O) is added to the flask and the mixture is stirred overnight at rt. The mixture is then filtered using a fluted filter paper, transferred to a round bottom flask, and placed on a rotary evaporator until the solvent fully evaporated. The resulting dried extract is transferred to a storage vial and stored at rt. 95% EtOH:H₂O is added to the E. rutaecarpa powder and the process is repeated 3-4 times. The combined dried extract is finely ground in a blender and stored at rt.

Macrocystis pyrifera Extraction Method

Macrocystis pyrifera powder is added to an Erlenmeyer flask equipped with a stir bar. 4:1 Ethanol:Water (EtOH:H₂O) is added to the flask and the mixture is stirred overnight at rt. The mixture is then filtered using a fluted filter paper, transferred to a round bottom flask, and placed on a rotary evaporator until the solvent fully evaporated. The resulting dried extract is transferred to a storage vial and stored at rt. 4:1 EtOH:H₂O is added to the Macrocystis pyrifera powder and the process is repeated 3-4 times. The combined dried extract is finely ground in a blender and stored at rt.

### Base Composition Formation

Macrocystis pyrifera and Evodia rutaecarpa extract (finely ground powder) are added to an Erlenmeyer flask equipped with a stir bar. Zemea® Propanediol (INCI name Propanediol) is added to the flask, with the final concentration of Macrocystis pyrifera extract ranging from 10-40 mg/mL (w/v) and Evodia rutaecarpa extract ranging from 5-30 mg/mL (w/v). The mixture is sonicated for 15 min at room temperature (rt), followed by stirring for 45 min at rt. The mixture is then filtered, using vacuum-assisted filtration with a Buchner funnel and grade 1 filter paper. Separately, the fucoidan composition is dissolved in distilled water, ranging from 5-20 mg/mL. The water-soluble fucoidan composition may be added to the propanediol solution up to 10% (v/v). Base compositions having various amounts of Evodia rutaecarpa extract, Macrocystis pyrifera extract, and Fucoidan composition were prepared as described in Table 1.

### Characterization of Base Composition

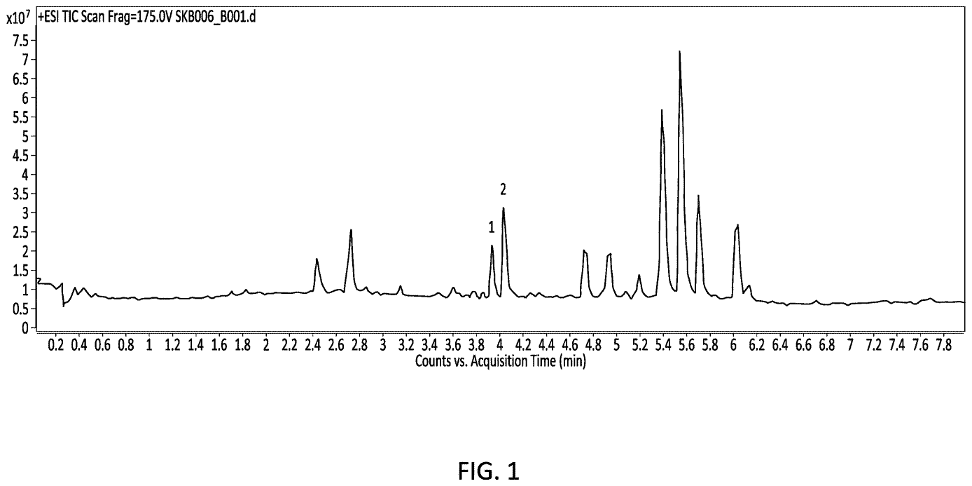

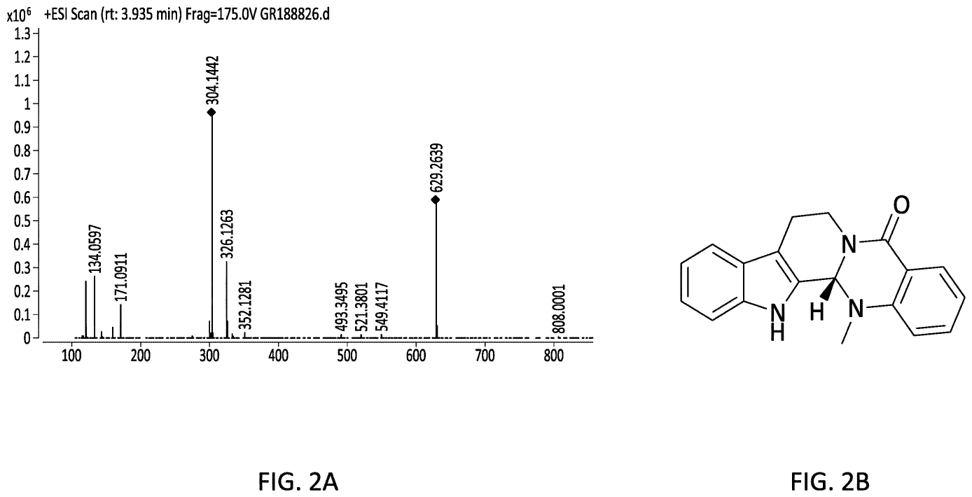

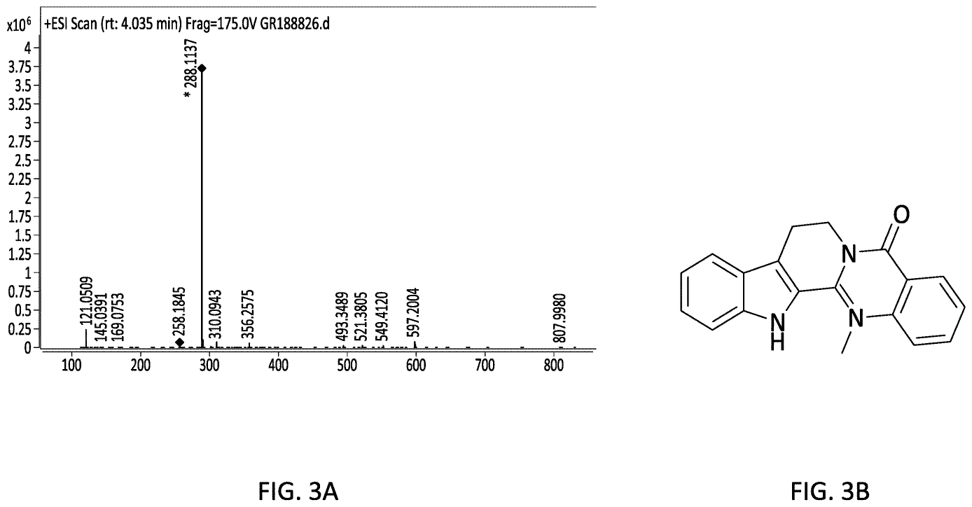

The compounds listed Table 2 are verified and quantified in the base composition using authentic standards with liquid chromatography-mass spectrometry (LCMS). Briefly, 75 mg of the base composition was diluted in LCMS-grade methanol (MeOH), vortexed, and filtered through a 0.2 μm syringe filter with a final concentration of 5 mg/mL. Authentic standards for rutaecarpine (Cayman Chemical, 22897), evodiamine (Cayman Chemical, 16885), and fucoxanthin (Millipore Sigma, 16337-5MG) were prepared as 10 mM stock solutions in DMSO. Each standard was diluted to 1 mM in LCMS-grade MeOH for analysis. All samples were analyzed using an Agilent UHPLC-QTOF 6538 system, equipped with a Phenomenex Kinetex Polar C18 column (100×2.1 mm, 2.6 μm). The mobile phases were water (H₂O, A) and acetonitrile (CH₃CN, B) with 0.1% formic acid, at a flow rate of 0.6 mL/min, and an elution gradient of 8-98% B over 6 minutes. FIG. 1 shows the mass spectral data for the base composition. Peak 1 was identified as evodiamine and Peak 2 was identified as rutaecarpine. FIG. 2A shows the mass spectral data for Peak 1. FIG. 2B shows the chemical structure of evodiamine. FIG. 3A shows the mass spectral data for Peak 2. FIG. 3B shows the chemical structure of rutaecarpine.

TABLE 2
List of Specific Compounds and Concentration Ranges in the Base Composition

| | Concentration | | Molecular | Exact | Observed | Retention |
|---|---|---|---|---|---|---|
| Compound | Range* | | Formula | Mass | m/z [M + H]⁺ | Time (min) |
| Rutaecarpine | 30-80 | mg/kg | C₁₈H₁₃N₃O | 287.1059 | 288.1137 | 4.03 |
| Evodiamine | 40-150 | mg/kg | C₁₉H₁₇N₃O | 303.1372 | 304.1442 | 3.94 |
| Fucoxanthin | 10-200 | mg/kg | C₄₂H₅₈O₆ | 658.4233 | 659.4359 | 5.93 |

*Concentration range of target compound in base composition

### Example 2. Antioxidant Activity of Base Composition in Stimulated Human Keratinocytes

The base composition and final formulation products that include the base composition are evaluated using an in vitro biological assay designed to assess their antioxidant, anti-inflammatory and protective effects, modeled on the assays in Wojciak et al., Int. J. Mol. Sci., 2022, 23, 13755 (https://doi.org/10.3390/ijms232213755), and described below using human keratinocytes and fibroblasts (ATCC CRL-2522™).

Transformed human keratinocyte (HaCaT) cells and human dermal fibroblasts are purchased from ATCC and cultured in Optimized Dulbecco's Modified Eagle's Medium (AddexBio, Catalog #C0003-02) supplemented with 10% Fetal Bovine Serum (Hyclone, Catalog #16140-071) and 1× Penicillin/Streptomycin (Gibco, Catalog #15140-122). When cells reach 80-90% confluency they are washed with 1×DPBS then detached from tissue culture flasks by incubation with 0.25% Trypsin (Gibco, Catalog #25200-056) for 3-5 minutes at 37 °C/5% CO₂. Cell culture media is added to trypsinized cells and then the cell suspension is transferred to a sterile 15 mL centrifuge tube to be spun down for 10 minutes at 1300 rpms. Media containing trypsin is aspirated from the cell pellet and then the cell pellet is re-suspended in 10 mL of cell culture media.

The antioxidant activity of the samples is analyzed using the DPPH free radical scavenging assay according to the method described by Brand-Williams et al., Food Sci. Technol., 1995, 28, 25 (https://doi.org/10.1016/S0023-6438(95)80008-5). 167 μL of 4 mM ethanol solution of DPPH is mixed with 33 μL of analyzed samples in different dilutions. The absorbance is measured at λ=516 nm every 5 min over 30 min using a UV-Vis spectrophotometer FilterMax F5 (Thermo Fisher). A DPPH solution mixed with an equal volume of distilled water is used as the control. The percentage of the DPPH radical scavenging is calculated using the equation:

% DPPH·scavenging=[(Abs control−Abs sample)/Abs control]×100%

### Example 3. Evaluation of Antioxidant Activity

H₂O₂ is used as a marker for the determination of detoxification power. This entity is part of the ROS (Reactive Oxygen Species) and considered as one of the most oxidizing non-radical species. When a sample is in the presence of hydrogen peroxide, all its antioxidant species spontaneously react with H₂O₂ and its quantity decreases. This depletion is directly influenced by the detoxification power and is quantified by an electrochemical sensor measuring oxidation of remaining H₂O₂. As the area of H₂O₂ oxidation peak is directly proportional to its concentration, one can compare the modulation of the peak area according to the test compound or the reference.

Stock solutions were prepared to have an initial sample concentration and a final composition of 33% of DMSO and 67% of either a hydrophilic (0.1 M PBS) or a lipophilic medium (0.1 M LiCl in ethanol). Stock solutions were then stirred for 30 seconds. Every sampling was performed under stirring conditions to maximize the homogenization of the diluted solutions. Two samples per work concentration were then generated by mixing 100 μL the stock solution either with 100 μL of the electrolytic solution and/or with 100 μL of the H₂O₂ solution.

A wait time of at least 30 minutes was observed to allow the reactions occur. Finally, 4 measurements per sample were performed with a work volume of 100 μL (1 for the blank test, 3 for the H₂O₂ treatment). The sample codes and their corresponding contents are as follows:
SKB001=Evodia rutaecarpa (ER) extract (10 mg/mL)
SKB004=Macrocystis pyrifera (MP) extract (10 mg/mL)
SKB009=Undaria pinnatifida (UP) extract (10 mg/mL)
SKB010=50% (5 mg/mL) of SKB001 and 50% (5 mg/mL) of SKB009
SKB011=38.5% (3.85 mg/mL) of SKB001, 38.5% (3.85 mg/ml) of SKB004, and 23% (2.3 mg/mL) of SKB009
SKB012=Aloe vera extract
SKB013=62.5% (6.25 mg/mL) of SKB001 and 37.5% (3.75 mg/mL) of SKB004.

In hydrophilic medium, all compositions exhibited an antioxidant activity between 14.5% and 82.9%. A moderate activity was highlighted with compositions SKB001, SKB009 and SKB010 (respectively 28.8%, 14.5% and 31.6%). Compositions SKB011, SKB012 and SKB013 showed a high activity and the results of composition SKB004 were even superior to the reference results. In lipophilic medium, SKB001 displayed a strongly high antioxidant activity with a value of 99.7%, close to that obtained using a vitamin C at 1 mg/ml as a reference. However, two major interferences were observed with SKB004 and SKB009, affecting the reliability of the calculated antioxidant activity. Also, the high concentration of DMSO in the assay medium increased the matrix effect on compositions SKB010 and SKB012, resulting in high variability with significant SEM values. Finally, no antioxidant activity was measured with SKB011 and SKB013. This lack of activity could also be explained by the DMSO effect, given that lipophilic mediums are more sensitive to matrix effects (of the samples themselves or their vehicle).

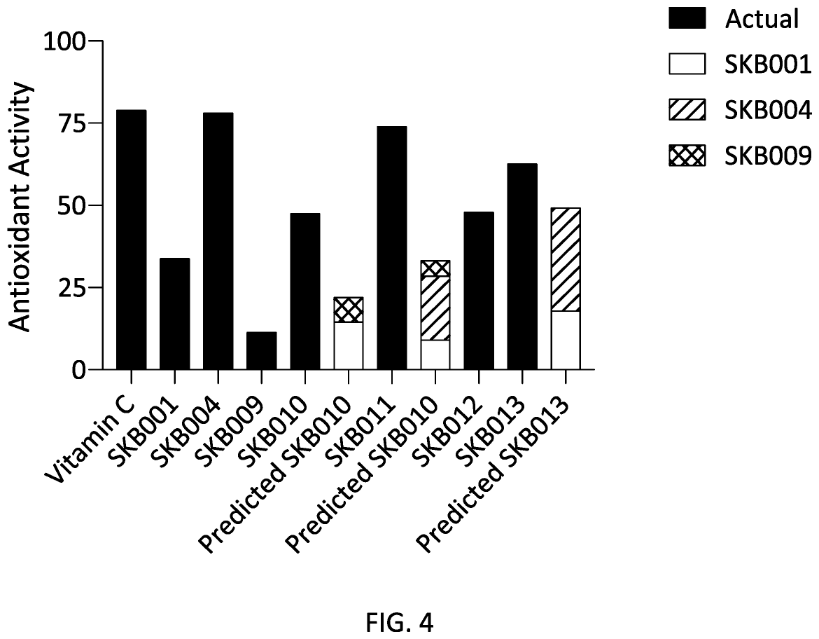

Results for these compounds in hydrophilic medium are shown in FIG. 4. The graph is normalized based on the individual extract responses at 10 mg/ml compared to the control (vitamin C at 1 mg/ml). The predicted values for compositions SKB010, SKB011, and SKB013 are based on the antioxidant activity of the individual extracts relative to their percentages within the mixture. Surprisingly, the actual activity of the combined extracts (all tested combinations, but specifically SKB011 which contains all three extracts) was significantly higher than predicted, demonstrating a synergistic antioxidant effect when the extracts were combined.

### Example 4. Effect of Base Composition on Cellular Growth and Viability

Human keratinocyte cultures are prepared as outlined in Example 2 and cell viability is assessed using the neutral red dye assay and the Alamar blue test.

The neutral red dye (Sigma Aldrich) assay is based on the initial protocol described by Borenfreund et al., Toxicol. Lett., 1985, 24, 119 (https://doi.org/10.1016/0378-4274(85)90046-3) and determines the accumulation of the neutral red dye in the lysosomes of viable, uninjured cells. The cells are placed in 96-well plates at a density of 1×10⁴ cells/well with fresh medium. After 24 h of pre-culture, the medium is aspirated, and varying concentrations (0.1, 0.5, 1, 2.5, and 5%) of the base composition will be added into each well and cultured for another 24 h. Unexposed cells are the control group. Following the exposure to the composition, the cells are incubated for 2 h with neutral red dye (40 μg/mL) dissolved in serum free medium (DMEM or MEM for HaCaT and fibroblasts, respectively). Next, the cells are washed with Phosphate Buffered Saline (PBS), and then 150 μL of a destain solution (EtOH/AcCOOH/H₂O₂, 50/1/49%) is added per well, followed by gentle shaking for 10 min until the neutral red dye is extracted from the cells and forms a homogenous solution. The Neutral Red (NR) dye uptake is determined by measuring the optical density (OD) of the eluted dye at 540 nm in a microtiter plate reader spectrophotometer FilterMax F5 (Molecular Devices, Silicon Valley, CA, US, Multi-Mode Analysis Software).

The Alamar Blue (AB) test is performed according to Page et al., Int. J. Oncol., 1993, 3, 473 (https://doi.org/10.3892/ijo.3.3.473) and measures resazurin sodium reduction. The cells are placed in 96-well plates at a density of 1×10⁴ cells/well with fresh medium. After 24 h of pre-culture, the medium is aspirated and varying dilutions (1:1000, 1:500, 1:100, 1:40, 1:20 v/v) of the tested samples are added into each well and cultured for another 24 h. Non-treated cells maintained in the cultured medium are the control group. After the exposure, a resazurin salt solution (Sigma, R7017) is transferred into the plates to a final volume 250 μL/well and a concentration of 60 μM in medium and incubated for 3 h at 37 °C in darkness. The fluorescence is measured with an excitation wavelength of 530 nm and an emission wavelength of 590 nm using a microplate reader (FilterMax F5, Molecular Devices, Silicon Valley, CA, US, Multi-Mode Analysis Software). All experiments are performed in triplicates for each base composition concentration and presented as a percentage of the control values (100%).

### Example 5. Anti-Inflammatory Activity of Base Composition

Human keratinocyte and fibroblast cultures are prepared as outlined in Example 2 and the immunomodulatory effect of the base composition on oxidative stress and inflammatory mediators is assessed as outlined below.

The cells are seeded in 96-well plates at a density of 1×10⁴ cells per well and initially cultured for 24 h before the experiment. Next, the culture medium is replaced with 10 μM H2DCFDA (Sigma Aldrich) in serum-free medium (DMEM or MEM for HaCaT and fibroblasts, respectively). The cells are incubated with H2DCFDA for 45 min before the treatment with the base composition. Then, the HaCaT and fibroblast cells are exposed to different dilutions of the composition (1:1000, 1:500, 1:100, 1:40, 1:20 v/v) for 45 min, and then treated with 1 mM hydrogen peroxide (H₂O₂). Cells treated with H₂O₂ alone are used as a positive control. Fibroblasts and HaCaT cells untreated with neither the test extract nor H₂O₂ are used as control cells. The DCF fluorescence, as a measure of oxidative stress, is measured after every 30 min for a total 90 min using a microplate reader FilterMax F5 (Molecular Devices, Silicon Valley, CA, US, Multi-Mode Analysis Software) at maximum excitation of 485 nm and emission spectra of 530 nm.

The levels of IL-6, IL-8, IL-10, and TNF-α (Elabscience, Houston, TX, USA) are used to assess the anti-inflammatory properties of the base composition and are measured immunoenzymatically (ELISA) using commercially available kits according to the manufacturer's instruction. The absorbance is measured using a microplate reader at 450 nm. A stock solution of standard compounds (Sigma Aldrich) is prepared using DMSO/culture medium (1:1) and diluted to the working concentration. The final concentration of DMSO does not exceed 0.1%, and this concentration does not affect the cell viability.

### Example 6. Immunomodulatory Activity of Base Composition on Gene Expression

Human keratinocyte and fibroblast cultures are prepared as outlined in Example 2 and the immunomodulatory effect of the base composition on oxidative stress and inflammatory mediators is assessed as outlined below.

For the gene expression assay, the cells are seeded on a 6-well plate at a density 5×10⁵ cells per well. After 24 h of pre-incubation, the cells are treated with the base composition and incubated for 24 h. Then, the samples are collected, and total RNA is extracted from the HaCaT and fibroblast cells using a EURx Universal RNA Purification Kit according to the manufacturer's protocol. The study is performed for all dilutions tested. The quality and quantity of the mRNA are determined spectrophotometrically at 260 and 280 nm (ND/1000 UV/VIS; Thermo Fisher NanoDrop). The reverse transcription (RT) reaction is performed at a final volume of 20 μL with 1 μg of RNA (as a cDNA template) using the High-Capacity cDNA Reverse Transcription Kit (Applied Biosystem™) according to the manufacturer's protocol. The study is performed for all dilutions tested. The RT-PCR is run using the Bio Rad C1000 Touch™ Thermal Cycler (Bio Rad Laboratories). The products obtained from the RT reaction are amplified using the TaqMan Gene Expression Master Mix (Life Technologies Applied Biosystems, USA) kit with TaqMan probes as primes for the specific genes encoding Nox-4 (assay ID Hs00276431_m1), SOD-1 (assay ID Hs00166575_m1), and GAPDH (assay ID Hs02786624_g1). Amplification is carried out in a total volume of 20 μL containing 1×TaqMan Gene Expression Master Mix and 1 μL of the RT product, which is used as the PCR template. The standard qPCR procedures are performed as follows: 2 min at 50 °C and 10 min at 95 °C, followed by 40 cycles of 15 s at 95 °C and 1 min at 60 °C using a Bio Rad CFX Connect™ Real-Time System (Bio Rad Laboratories). The mRNA expression is calculated relative to a nontargeting control in each experiment. The experiments are repeated in triplicate. The level of expression of the gene of interest is calculated using the comparative threshold cycle (Ct) method of Livak et al., Methods, 2001, 25, 402 (https://doi.org/10.1006/meth.2001.1262). GADPH is used as the reference gene.

### Example 7. IL-8 Release by UV-Irradiated Normal Human Epidermal Keratinocytes (NHEKs)

Test compositions were evaluated for their effect on release of inflammatory markers in UV-irradiated normal human epidermal keratinocytes. Samples were prepared and tested at the concentrations indicated in Table 3.

TABLE 3

| Sample | | Stock Solution | Test Concentration |
|---|---|---|---|
| Code | Contents | (mg/ml in DMSO) | (mg/ml) |
| SKB001 | ER | 100 | 1.7 × 10⁻⁴ and 5 × 10⁻⁴ |
| SKB004 | MP | 60 | 0.003 and 0.008 |
| SKB009 | UP | 100 | 1.3 × 10⁻⁴ and 4 × 10⁻⁴ |
| SKB010 | 50% ER + 50% UP | 200 | 3 × 10⁻⁴ and 9.1 × 10⁻⁴ |
| SKB011 | 38.5% ER + 38.5% MP + | 260 | 3.3 × 10⁻⁴ and 0.001 |
| | 23% UP | | |
| SKB012 | Aloe vera | 500 | 0.021 and 0.062 |
| SKB013 | 62.5% ER + 37.5% MP | 160 | 2.4 × 10⁻⁴ and 7.3 × 10⁻⁴ |

ER = Evodia rutaecarpa extract
MP = Macrocystis pyrifera extract
UP = Undaria pinnatifida extract

Keratinocytes were seeded in 96-well plates and cultured for 24 hours in culture medium (Keratinocyte-SFM, supplemented with Epidermal Growth Factor and Pituitary extract) and for a further 24 hours in assay medium. The culture conditions were 37 °C and 5% CO₂. The medium was then replaced by assay medium containing or not (control) the test composition. The cells were then irradiated with UVB—275 mJ/cm² (+UVA—1.9 J/cm²) (in absence of the test composition). The lamp used was a SOL500 Sun Simulator equipped with an H2 filter (Dr. Hönle, AG). A non-irradiated control condition was performed in parallel. After irradiation, the medium was replaced by assay medium containing or not (irradiated control) the test composition and the cells were incubated for 48 hours. All experimental conditions were performed in n=3.

Inflammatory markers released in the medium were measured using a specific Protein Array kit (Proteome Profiler™, Human Cytokine Array Kit, R&D Systems, ref. ARY005B) according to the supplier's instructions. The protein array membrane was analyzed by densitometry of the different spots using ImageQuant 800 (Cytiva) and ImageQuantTL software (Cytiva).

The results are provided in Table 4. The table lists fold-changes for the tested conditions relative to a reference value. For the irradiated control, the fold-change are relative to the non-irradiated control. The remaining fold-changes are relative to the stimulated control.

Exposure to UV irradiation of untreated keratinocytes induced a strong release of IL-8 (9.76 fold). This stress also induced the release of Complement Component C5/C5a (4.12 fold), CXCL1/GROα (5.09 fold), IL-1α/IL-1F1 (4.53 fold), IL-1β/IL-1F2 (2.32 fold) and IL-6 (2.85 fold). A decrease of TREM-1 release (0.49 fold) was also observed.

Under the experimental conditions of the assay, compositions SKB001, SKB004 and SKB009 did not significantly modulate the secretory profile of the irradiated keratinocytes, and they did not show a curative effect on these cells. In contrast, compositions SKB010, SKB011, SKB012 and SKB013 displayed curative effects against UV irradiation in keratinocytes.

Composition SKB010 demonstrated inhibition of Complement Component C5/C5a, IL-1α/IL-1F1, IL-6, and IL-8. These results were associated with an inhibition of the release of MIP-1α/MIP-1β, CCL5/RANTES, CD40 Ligand/TNFSF5, G-CSF, GM-CSF, ICAM-1/CD54, IFN-γ, IL-10, IL-17E, IL-18/IL-1F4 and IL-32α.

Composition SKB011 demonstrated inhibition of IL-1α/IL-1F1, IL-1β/IL-1F2, IL-6, and IL-8. No effect was observed on Complement Component C5/C5a, CXCL1/GROα and TREM-1 release by the irradiated keratinocytes. These results were associated with an inhibition of the release of GM-CSF, ICAM-1/CD54, IFN-γ, IL-18/IL-1F4, IL-21, IL-27 and IL-32α.

Composition SKB012 demonstrated inhibition of Complement Component C5/C5a, CXCL1/GROα, IL-1α/IL-1F1, IL-1β/IL-1F2, and IL-6. No effect was observed on IL-8, whereas TREM-1 release was decreased. These results were associated with an inhibition of the release of CCL2/MCP-1, MIP-1α/MIP-1β, CCL5/RANTES, CD40 Ligand/TNFSF5, CXCL12/SDF-1, G-CSF, GM-CSF, ICAM-1/CD54, IFN-γ, IL-4, IL-5, IL-10, IL-17E, IL-18/IL-1F4, IL-21, IL-27 and IL-32α.

Composition SKB013 demonstrated inhibition of Complement Component C5/C5a, CXCL1/GROα, IL-1α/IL-1F1, IL-1β/IL-1F2, IL-6 and of IL-8. No effect was observed on TREM-1 release. These results were associated with an inhibition of the release of MIP-1α/MIP-1β, CCL5/RANTES, CD40 Ligand/TNFSF5, CXCL11/I-TAC, CXCL12/SDF-1, G-CSF, GM-CSF, ICAM-1/CD54, IFN-γ, IL-5, IL-10, IL-17A, IL-17E, IL-18/IL-1F4, IL-21, IL-27, IL-32α, MIF and TNF-α.

Surprisingly, the combination compositions SKB010, SKB011, and SKB013 displayed curative effects, whereas the singe agent compositions SKB001, SKB004 and SKB009 did not. Thus, the combination compositions demonstrated remarkable synergism.

TABLE 4

| | | | Irradiated conditions: UVB − 275 mJ/cm² (+UVA − 2.1 J/cm²) | | | | | | | | | | | | | |
|---|---|---|---|---|---|---|---|---|---|---|---|---|---|---|---|---|
| | | Non- | SKB001 | | SKB004 | | SKB009 | | SKB010 | | SKB011 | | SKB012 | | SKB013 | |
| | Irradiated | irradiated | 1.67 × 10⁻⁴ | 5 × 10⁻⁴ | 2.67 × 10⁻³ | 0.008 | 1.33 × 10⁻⁴ | 4 × 10−4 | 3.03 × 10⁻⁴ | 9.1 × 10⁻⁴ | 3.33 × 10⁻⁴ | 0.001 | 0.021 | 0.062 | 2.43 × 10⁻⁴ | 7.3 × 10⁻⁴ |
| Analyte | control | control | mg/ml | mg/ml | mg/ml | mg/ml | mg/ml | mg/ml | mg/ml | mg/ml | mg/ml | mg/ml | mg/ml | mg/ml | mg/ml | mg/ml |
| CCL1/I-309 | 1.45 | 1.39 | 1.00 | 1.20 | 0.98 | 1.42 | 1.07 | 1.19 | 0.94 | 1.06 | 0.93 | 1.17 | 0.64 | 1.18 | 0.65 | 1.02 |
| CCL2/MCP-1 | 1.85 | 1.40 | 0.85 | 0.97 | 1.00 | 1.12 | 1.02 | 1.33 | 0.72 | 0.89 | 0.81 | 0.97 | 0.32 | 0.77 | 0.53 | 0.95 |
| MIP-1α/MIP-1β | 2.00 | 1.55 | 0.74 | 0.84 | 0.87 | 1.02 | 0.80 | 1.10 | 0.36 | 0.82 | 0.55 | 0.74 | 0.22 | 0.85 | 0.32 | 0.77 |
| CCL5/RANTES | 1.23 | 1.69 | 0.64 | 0.84 | 1.08 | 0.67 | 0.51 | 1.45 | 0.34 | 0.65 | 0.51 | 0.67 | 0.37 | 0.77 | 0.44 | 0.66 |
| CD40 | 0.19 | 1.54 | 0.61 | 1.25 | 1.75 | 0.76 | 0.96 | 1.56 | 0.22 | 0.64 | 0.54 | 0.76 | 0.32 | 0.99 | 0.34 | 0.97 |
| Ligand/TNFSF5 | | | | | | | | | | | | | | | | |
| Complement | 0.18 | 0.24 | 0.62 | 1.27 | 1.47 | 0.64 | 0.51 | 1.07 | 0.43 | 0.60 | 0.50 | 0.55 | 0.15 | 0.75 | 0.10 | 0.29 |
| Component | | | | | | | | | | | | | | | | |
| C5/C5a | | | | | | | | | | | | | | | | |
| CXCL1/GROα | 0.94 | 0.20 | 1.14 | 1.08 | 0.95 | 1.02 | 0.92 | 0.81 | 0.67 | 0.64 | 0.60 | 0.60 | 0.40 | 0.55 | 0.58 | 0.40 |
| CXCL10/IP-10 | 1.21 | 1.97 | 1.88 | 2.10 | 1.65 | 2.22 | 1.69 | 1.67 | 1.72 | 1.96 | 1.66 | 1.29 | 0.51 | 1.17 | 1.50 | 0.93 |
| CXCL11/I-TAC | 1.96 | 1.19 | 0.98 | 0.98 | 0.99 | 1.17 | 0.97 | 1.04 | 0.79 | 0.94 | 0.72 | 0.85 | 0.57 | 0.80 | 0.47 | 0.64 |
| CXCL12/SDF-1 | 1.73 | 1.47 | 0.78 | 0.75 | 0.90 | 1.10 | 0.79 | 0.73 | 0.80 | 0.92 | 0.71 | 0.66 | 0.44 | 0.61 | 0.46 | 0.50 |
| G-CSF | 0.73 | 1.37 | 0.65 | 0.79 | 0.81 | 0.87 | 0.68 | 0.88 | 0.41 | 0.66 | 0.63 | 0.66 | 0.26 | 0.49 | 0.31 | 0.53 |
| GM-CSF | 0.85 | 0.84 | 0.99 | 1.15 | 0.90 | 1.12 | 0.61 | 0.71 | 0.33 | 0.40 | 0.50 | 0.33 | 0.29 | 0.54 | 0.33 | 0.46 |
| ICAM-1/CD54 | 0.52 | 1.11 | 0.60 | 1.31 | 1.34 | 1.06 | 0.82 | 1.22 | 0.43 | 0.43 | 0.42 | 0.65 | 0.27 | 0.59 | 0.25 | 0.73 |
| IFN-γ | 0.22 | 0.57 | 0.87 | 1.09 | 1.30 | 1.11 | 0.61 | 1.06 | 0.51 | 0.43 | 0.56 | 0.45 | 0.39 | 0.61 | 0.37 | 0.51 |
| IL-1α/IL-1F1 | 0.35 | 0.22 | 0.87 | 0.98 | 0.70 | 0.88 | 0.50 | 0.69 | 0.47 | 0.55 | 0.40 | 0.48 | 0.16 | 0.27 | 0.31 | 0.21 |
| IL-1β/IL-1F2 | 1.07 | 0.43 | 1.03 | 1.24 | 0.97 | 0.82 | 0.58 | 0.86 | 0.61 | 0.59 | 0.49 | 0.41 | 0.19 | 0.36 | 0.57 | 0.30 |
| IL-1ra/IL-1F3 | 1.42 | 0.89 | 1.00 | 0.84 | 0.97 | 1.13 | 1.02 | 0.84 | 0.77 | 0.95 | 0.65 | 0.75 | 0.54 | 0.58 | 0.43 | 0.45 |
| IL-2 | 2.04 | 1.23 | 0.84 | 0.86 | 0.95 | 1.06 | 0.93 | 1.02 | 0.77 | 0.91 | 0.74 | 0.81 | 0.51 | 0.75 | 0.54 | 0.64 |
| IL-4 | 1.39 | 1.36 | 0.80 | 0.67 | 0.80 | 0.69 | 0.65 | 1.16 | 0.63 | 0.70 | 0.66 | 0.82 | 0.40 | 0.68 | 0.51 | 0.67 |
| IL-5 | 0.20 | 1.19 | 0.72 | 0.86 | 0.94 | 0.69 | 0.68 | 1.41 | 0.56 | 0.55 | 0.78 | 0.92 | 0.24 | 0.56 | 0.45 | 0.77 |
| IL-6 | 0.08 | 0.35 | 1.22 | 1.80 | 1.39 | 2.15 | 0.87 | 1.15 | 0.47 | 0.68 | 0.49 | 0.50 | 0.35 | 0.54 | 0.29 | 0.39 |
| IL-8 | 0.36 | 0.10 | 1.21 | 1.21 | 1.15 | 1.49 | 0.79 | 0.91 | 0.44 | 0.43 | 0.48 | 0.33 | 0.53 | 0.60 | 0.38 | 0.35 |
| IL-10 | 0.66 | 0.72 | 0.93 | 1.98 | 1.72 | 0.93 | 1.21 | 1.97 | 0.49 | 0.77 | 0.57 | 0.69 | 0.17 | 0.54 | 0.28 | 0.46 |
| IL-12 p70 | 1.18 | 1.35 | 1.46 | 2.05 | 1.70 | 1.30 | 1.21 | 2.04 | 0.97 | 1.18 | 0.97 | 0.82 | — | 0.69 | 0.82 | 0.95 |
| IL-13 | 1.62 | 1.19 | 1.10 | 1.00 | 1.09 | 1.19 | 1.06 | 1.04 | 1.09 | 1.01 | 0.86 | 0.94 | 0.79 | 0.90 | 0.58 | 0.69 |
| IL-16 | 2.17 | 1.31 | 0.97 | 0.81 | 0.80 | 0.92 | 0.81 | 0.79 | 0.86 | 0.87 | 0.70 | 0.84 | 0.68 | 0.73 | 0.57 | 0.61 |
| IL-17A | 2.18 | 1.47 | 0.86 | 0.67 | 0.89 | 0.95 | 0.81 | 1.16 | 0.88 | 0.77 | 0.83 | 0.76 | 0.65 | 0.69 | 0.49 | 0.60 |
| IL-17E | 1.20 | 1.64 | 0.85 | 0.75 | 0.81 | 0.76 | 0.55 | 0.86 | 0.43 | 0.53 | 0.62 | 0.52 | 0.30 | 0.50 | 0.37 | 0.36 |
| IL-18/IL-1F4 | 0.78 | 1.21 | 0.98 | 1.01 | 0.85 | 0.72 | 0.60 | 0.62 | 0.49 | 0.37 | 0.36 | 0.41 | 0.36 | 0.40 | 0.39 | 0.37 |
| IL-21 | 0.89 | 0.99 | 0.98 | 1.26 | 1.11 | 0.94 | 0.71 | 1.04 | 0.57 | 0.50 | 0.48 | 0.46 | 0.24 | 0.44 | 0.40 | 0.38 |
| IL-27 | 1.64 | 1.16 | 0.45 | 1.30 | 1.04 | 0.84 | 0.60 | 1.13 | 0.53 | 0.60 | 0.42 | 0.30 | 0.08 | 0.59 | 0.23 | 0.27 |
| IL-32α | 0.88 | 1.35 | 0.64 | 0.82 | 0.77 | 0.42 | 0.60 | 0.95 | 0.45 | 0.49 | 0.54 | 0.46 | 0.02 | 0.51 | 0.29 | 0.21 |
| MIF | 1.34 | 0.69 | 1.03 | 0.79 | 0.96 | 0.96 | 0.99 | 0.79 | 0.94 | 0.93 | 0.76 | 0.80 | 0.62 | 0.79 | 0.45 | 0.53 |
| Serpin E1/PAI-1 | 2.49 | 1.11 | 0.94 | 0.82 | 0.87 | 0.91 | 0.93 | 0.69 | 0.88 | 0.87 | 0.77 | 0.83 | 0.71 | 0.80 | 0.66 | 0.69 |
| TNF-α | 2.26 | 1.74 | 0.69 | 0.70 | 0.78 | 0.82 | 0.73 | 0.95 | 0.68 | 0.62 | 0.67 | 0.60 | 0.57 | 0.61 | 0.45 | 0.52 |
| TREM-1 | — | 2.06 | 0.64 | 0.91 | 1.11 | 0.75 | 0.76 | 1.21 | 0.63 | 0.68 | 0.78 | 0.70 | 0.08 | 0.69 | 0.58 | 0.65 |

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1-92. (canceled)

93. A composition comprising an extract from a fucoidan-containing seaweed and Evodia rutaecarpa extract, wherein the fucoidan-containing seaweed is selected from the group consisting of: Macrocystis pyrifera and Undaria pinnatifida.

94. The composition of claim 93, further comprising an aloe extract.

95. The composition of claim 93, wherein the fucoidan-containing seaweed is Macrocystis pyrifera.

96. The composition of claim 93, further comprising additional fucoidan.

97. The composition of claim 95, wherein the Macrocystis pyrifera extract is present in an amount between about 0.1% w/w and about 10% w/w.

98. The composition of claim 95, wherein the Macrocystis pyrifera extract is present in an amount between about 1% w/w and about 10% w/w.

99. The composition of claim 93, wherein the Evodia rutaecarpa extract is present in an amount between about 0.1% w/w and about 20% w/w.

100. The composition of claim 93, wherein the Evodia rutaecarpa extract comprises a solution in propane-1,3-diol.

101. The composition of claim 96, wherein the additional fucoidan is in an extract from a seaweed present in an amount between about 2% w/w and about 20% w/w.

102. The composition of claim 93, wherein rutaecarpine is present in an amount between about 15 mg/kg to about 200 mg/kg.

103. The composition of claim 93, wherein evodiamine is present in an amount between about 20 mg/kg to about 300 mg/kg.

104. The composition of claim 95, wherein fucoxanthin is present in an amount between about 5 mg/kg to about 400 mg/kg.

105. The composition of claim 93, wherein the composition is in the form of: lotion, spray, cream, serum, aqueous solution, paste, jelly, wax, oil, semi-solid gel ointment, gel-cream, lip balm, or multi-purpose balm.

106. The composition of claim 93, further comprising one or more of: propane-1,3-diol, shea butter, cocoa butter, olive oil, coconut oil, avocado oil, dimethicone, petroleum jelly, glycerin, ceramide, honey, buttermilk, castor oil, aloe extract, aloe vera gel, aloe butter, argan oil, caprylic/capric triglycerides, flaxseed oil, jojoba oil, rose him seed oil, safflower oil, sunflower oil, sea buckthorn oil, red raspberry seed oil, squalane, niacinamide, mango butter, kokum butter, almond butter, apricot kernel butter, hemp butter, macadamia nut butter, sal butter, beeswax, black cumin seed oil, vitamin E oil, murumuru butter, cupuacu butter, tamanu oil, meadowfoam seed oil, candelilla wax, carnauba wax, laurel wax, sunflower seed wax, and Cera bellina.

107. The composition of claim 93, further comprising propane-1,3-diol.

108. The composition of claim 93, further comprising about 15% w/w aloe extract.

109. The composition of claim 93, further comprising about 0.5% w/w to about 5% w/w sea buckthorn oil.

110. The composition of claim 93, further comprising about 0.5% w/w to about 10% w/w squalane.

111. The composition of claim 93, further comprising about 3% w/w to about 5% w/w red raspberry seed oil.

112. The composition of claim 93, further comprising about 1% w/w to about 10% w/w niacinamide.

113. The composition of claim 93, wherein the composition further comprises about 0.5% w/w to about 5% w/w panthenol.

114. A method of reducing oxidative stress, comprising administering the composition of claim 93 to the skin of a subject.

115. A method of reducing UV-induced inflammation, comprising administering the composition of claim 93 to the skin of a subject.

116. The composition of claim 93, wherein the composition comprises a moisturizer.

117. The composition of claim 93, wherein the composition comprises a balm.
